(19) United States
(12) Patent Application Publication
(10) Pub. No.: US 2007/0118078 A1
(43) Pub. Date: May 24, 2007

(54) Method and apparatus for controlled feeding of an infant

(76) Inventors: David J. McNally, Salt Lake City, UT (US); Charity Williams, Bountiful, UT (US); Phil Eggers, Salt Lake City, UT (US); Chris Steinman, Salt Lake City, UT (US); Shawn Fojtik, Park City, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 11/283,404

(22) Filed: Nov. 18, 2005

(51) Int. Cl. A61M 37/00; A61M 1/06; A61M 31/00

(52) U.S. Cl. 604/131; 604/500; 604/74

(57) ABSTRACT

A method and apparatus for controlled feeding of an infant provides a safer and less expensive apparatus for the enteral feeding of a neonatal infant, as well as a mechanism for minimizing separation of fluid for any enteral feeding patient. The apparatus allows for dispensing the feeding solution from conventional baby bottles or breast pump reservoirs, or other convenient reservoirs in combination with a peristaltic pump. The feeding system helps reduce the risk for contaminating the feeding solution by minimizing the handling of the feeding solution. By utilizing a pump which is less expensive than current neonatal feeding pumps, the feeding system is more cost effective in all environments and more suitable for home use.

### BACKGROUND OF THE INVENTION

1. The Field of the Invention

The present invention relates to a method and apparatus for controlled feeding of a neonatal or pediatric infant. More specifically, the present invention relates to the use of an enteral feeding pump for gastroinstestinal or nasoenteric enteral feeding of an infant and relates to a variety of containers which can be used to improve neonatal or pediatric feeding. The present invention also relates to a method for improving the delivery of enteral feeding solutions for patient's of all ages.

2. State of the Art

Many infants which are born prematurely or which are smaller or underdeveloped do not have sufficient mouth strength to feed normally. That is to say that many premature infants do not have sufficient strength to breast feed or to draw milk from a bottle. For some infants, it is sufficient to place an nasoenteric feeding tube and allow gravity to feed the breast milk or other feeding solution to the child.

Other infants, however, are not able to handle a large dose of feeding solution in a relatively short amount of time. These infants are typically fed using a feeding pump which pumps the milk, etc. through a nasal feeding tube which has been placed through the nose and into the stomach or through an enteral feeding tube placed in a stoma in the stomach wall. Due to their size, many neonatal infants require very slow administration of feeding solution, on the order of 1 mL per hour. In some cases, the desired administration rate may be as low as 0.1 mL per hour. There is thus a need for a neonatal feeding pump and system which is capable of delivering a feeding solution at a slow rate, and which is very precise.

Currently, syringe pumps are most often used to feed such neonatal infants. Available peristaltic pumps are typically designed for the enteral feeding of adults and children and generally pump the solution too fast, and are not accurate enough for neonatal use. Thus, syringe pumps are used. Syringe pumps are also commonly used for introducing medication into an I.V. line for administration to a patient. Thus, the syringe pumps are very accurate and designed for low flow rates. When syringe pumps are used for neonatal feeding, it is necessary to transfer breast milk from the breast pump reservoir to a syringe prior to delivery to an infant, increasing the risk of contaminating the milk and the complexity of the feeding procedure. Additionally, syringe pumps are often more expensive than other types of pumps, such as peristaltic pumps, making them impractical for home use and increasing the cost of owning these pumps for a hospital.

The expense of the syringe pumps makes them generally unavailable for home use and requires that the neonatal infant remain in the hospital until the infant is capable of feeding from a bottle or breast feeding, or is capable of receiving the higher volume of feeding solution delivered from a typical enteral feeding pump. This can increase the time that the infant is in the hospital, also increasing the cost of the hospital stay.

Yet another concern with the delivery of feeding fluid to an infant is separation within the fluid. In most situations, the food of choice for a neonatal infant is breast milk. However, because relatively small quantities of the breast milk are being delivered, a feeding container with a substantial amount of breast milk will have time for the breast milk to separate. Needed fats will separate out from the milk and rise, leaving a watery mixture of proteins and sugars. This can provide inconsistent nutrients to the infant. Additionally, because such small doses of the milk are administered, a large container of milk may have time to spoil or culture bacterial growth. Furthermore, some conventional feeding sets can include 15 ml of solution withing the feeding set alone, thereby increasing the risk of spoilage or waste of precious feeding solution—such as breast milk. Thus, it is desirable to provide containers which contain relatively small amounts of breast milk or other feeding solutions. Likewise, it is desirable to minimize the risk of, separation which could result in some doses of the milk being watery proteins and sugars, while other doses are principally milk fats.

In addition to the above, there is a need to keep safety at the forefront when feeding the neonatal infant. Safety concerns with syringe feeding pumps include the possibility that feeding solution may be administered through an intravenous line, leading to the possible harm or even death of the patient. Enteral feeding set are typically formed with non-IV compatible adaptors for connection to feeding tubes. If tubing connections are not propery controlled, the tubing from one system (such as feeding) may inadvertently become confused or tangled with tubing from another system, such as that designed for IV medictions.

There is thus a need for a neonatal feeding system which is easier and more convenient to use, and which provides increase control, facilitates lower storage volumes and increased safety. There is also a need for a neonatal feeding system which is less expensive than the currently available syringe pumps used for feeding. A neonatal feeding system which is easier to use, safer and less expensive may allow parents to take premature infants home from the hospital earlier.

### SUMMARY OF THE INVENTION

It is an object of the present invention to provide an improved feeding apparatus and method of use for controlled feeding of infants. According to one aspect of the present invention, a feeding system is provided which is easier to use, safer and provides more reliable nutrition to the infant. This can be accomplished in several ways including reduced storage volumes, more consistent solution delivery, and reduced risk of error in connection with the patient's feeding tube.

In accordance with one aspect of the invention, the feeding system may be designed such that the feeding solution reservoir is usable with breast pumps, allowing the milk to be delivered to an infant without unnecessary transfer of the milk between containers. If desired, an insert or adapter may be provided which allows conventional bottles or breast pump reservoirs to be connected to the tubing of an enteral feeding set and thereby to the pump for delivery to an infant. As such the risk of contamination is decreased and the cost of the system is reduced.

According to another aspect of the present invention, a feeding solution reservoir is provided which prevents the formation of a partial vacuum inside of the reservoir as solution is drawn from the reservoir. A feeding solution reservoir may be collapsible as the feeding solution is drawn from the reservoir, preventing a partial vacuum inside of the reservoir without requiring a vent. Alternatively, a vent may be provided whereby air is allowed to enter the reservoir as the solution is drawn from the reservoir. Such a configuration reduces the amount of work necessary for a pump, such as a peristaltic enteral feeding pump, to draw the breast milk or other feeding solution from the container for delivery to the child. Those skilled in the art will appreciate that if the container/reservoir is a syringe, most enteral feeding pumps will not generate sufficient suction on the upstream or inlet portion of the infusion set to reliably draw the breast milk or other feeding solution from the syringe.

According to another aspect of the present invention, an infusion set may be provided which is designed for the feeding of neonatal infants. The infusion set may have a distal end thereof which is tapered or which is formed in a size and shape suitable for nasal feeding of a neonatal infant. Additionally, an infusion set may be provided which has an adapter configured for attachment directly to a feeding reservoir, such as a bottle or breast pump reservoir.

In accordance with another aspect of the present invention, the storage container or reservoir may be very small, such as a syringe, and be provided with affirmative force, such as a spring or elastomeric biasing element, to assist the pump in drawing the feeding solution from the container.

In accordance with yet another aspect of the present invention, the storage container is configured to minimize the overall separation of the breast milk or other feeding solution within the container. This can be done by utilizing a syringe in conjunction with an enternal feeding pump, or by utilizing custom containers which minimize overall separation.

In addition to concerns regarding the nutritional nature of each dose of feeding solution received by the child, another concern in enteral feeding can be separation of the feeding solution and medicine or other solutions mixed therewith. For some infants and even some children and adults, it is necessary to mix medicines into the feeding solution for delivery to the patient's digestive system. Because some medicines, etc., have different densities than the feeding solution, or my be less soluble in the feeding solution, separation can be a problem. In some extreme cases, parents or patients are required to wake every hour and shake the solution container to ensure that the medicine is not separating out of the solution. Failure to do so can result in inadequate doses of the medicine, followed by excessive doses. Thus, minimizing separation not only improves nutritional solution delivery, it can also be critical to proper medicinal therapy.

It will be appreciated that the various aspects of the invention may not all be found present in embodiments which are made in accordance with the individual aspects of the invention and there is no requirement that any embodiment containing one or more aspects of the present invention include other aspects of the present invention. Rather, the claims are drawn to the various aspects of the invention and should not be viewed as requiring elements or aspects not set forth specifically therein.

### BRIEF DESCRIPTION OF THE DRAWINGS

Various embodiments of the present invention are shown and described in reference to the numbered drawings wherein:

FIG. 1 shows a perspective view of a neonatal feeding pump as is known in the prior art;

FIG. 2 shows a perspective view of a neonatal feeding system according to the present invention;

FIG. 3 shows a side view of a feeding solution reservoir according to the present invention;

FIG. 4 shows a side view of an insert for connecting a bottle to an infusion set according to the present invention;

FIG. 5 shows a side view of another feeding solution reservoir according to the present invention;

FIG. 6 shows a side view of an adapter according to the present invention;

FIG. 7 shows a side view of another adapter according to the present invention;

FIG. 8 shows a side view of another feeding solution reservoir according to the present invention;

FIG. 9A shows a side view of a syringe being filled in accordance with the present invention;

FIG. 9B shows the syringe of FIG. 9A being attached to the infusion set configured for use with a peristaltic enteral feeding pump;

FIG. 9C shows the infusion set being loaded into an enteral feeding pump;

FIG. 9D shows the infusion set and pump being attached to a custom bracket or mount to facilitate delivery of the breast milk or other feeding solution from the syringe to an infant;

FIG. 10A shows a front view of an enteral feeding pump and feeding solution reservoir in accordance with another aspect of the present invention;

FIG. 10B shows a top view of the pump and feeding solution reservoir of FIG. 10A;

FIG. 10C shows an end view of the pump and feeding solution reservoir of FIGs. 10A and 10B.

FIG. 11A shows a front view of an enteral feeding pump and feeding solution reservoir in accordance with another aspect of the present invention;

FIG. 11B shows a top view of the pump and feeding solution reservoir of FIG. 11A;

FIG. 11C shows an end view of the pump and feeding solution reservoir of FIGs. 11A and 11B;

FIG. 12A shows a front view of an enteral feeding pump and feeding solution reservoir in accordance with another aspect of the present invention;

FIG. 12B shows a top view of the pump and feeding solution reservoir of FIG. 12A;

FIG. 12C shows an end view of the pump and feeding solution reservoir of FIGS. 12A and 12B;

FIG. 13 shows a side view of an alternate configuration of a solution container in accordance with the principles of the present invention;

FIG. 14 shows a front view of an enteral feeding pump similar to that of FIG. 12A with a shaking mechanism for minimizing fluid separation.

FIG. 15A shows a cross-section view of an alternate configuration of a solution container designed to minimize separation within the enteral feeding solution prior to delivery to the patient;

FIG. 15B shows a side view of the solution container of FIG. 14A;

FIG. 15C shows an end view of the solution container disposed on a rocker motor;

FIG. 16A shows a side view of an alternate configuration of a solution container designed minimize separation within the feeding solution;

FIG. 16B shows another view of the solution container of FIG. 16A, wherein the pump is mixing the contents of the container.

FIG. 17A shows an alternate embodiment of a solution container for minimizing separation of solution;

FIGS. 17B, 17C and 17D show additional details of and alternate positions of the solution container in FIG. 17; and

FIGS. 18A and 18B show static mixers for minimizing fluid separation within the enteral feeding solution.

It will be appreciated that the drawings are illustrative and not limiting of the scope of the invention which is defined by the appended claims. The various embodiments shown accomplish various aspects and objects of the invention, and no one embodiment need accomplish all aspects of the invention.

### DETAILED DESCRIPTION

The drawings will now be discussed in reference to the numerals provided thereon so as to enable one skilled in the art to practice the present invention. The drawings and descriptions are exemplary of various aspects of the invention and are not intended to narrow the scope of the appended claims. Nor is it suggested that any embodiment of one aspect of the invention must include other aspects discussed herein.

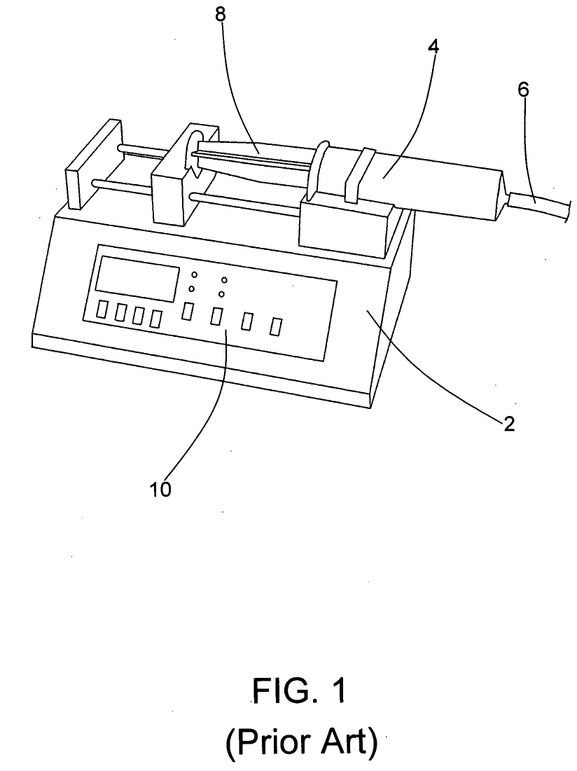

Turning to FIG. 1, a perspective view of a feeding system known in the prior art is shown. The feeding system uses a syringe pump 2 to provide feeding solution to a neonatal infant. As previously mentioned, syringe pumps 2 are sufficiently accurate and provide a low enough flow rate to be useful for neonatal feeding. Syringe pumps 2 often are, however, expensive. This limits the availability of the pumps 2, inhibiting smaller institutions and individual persons from owning such pumps. Additionally, once the child grows, the child will typically be transitioned to an enteral feeding pump as he or she grows if enteral feeding is still necessary.

In the prior art configuration, a syringe 4 is filled with feeding solution, which is commonly breast milk. The use of a syringe requires the caregiver to transfer the feeding solution from a container holding the solution into the syringe 4.

It will be appreciated by those of skill in the art that the availability of breast milk creates potential complications. Many neonatal infants must stay in an intensive care unit for a prolonged period of time. Thus, it is common for the mother of the infant to be released long before the infant. In such situations, the infant may require milk at numerous times that the mother is not present. Thus, it is common for the mother to use a breast pump and to bring in a certain quantity of breast milk which may last for 12 hours or more. To minimize the risk of bacterial growth, any breast milk not being immediately used is kept in a refrigerator. Additionally, the larger quantity of breast milk allows for small doses to be given to the child at any time needed.

The filled syringe 4 is connected to tubing 6 which delivers the solution to the infant (not shown). The syringe 4 is then mounted in the pump 2, which holds the syringe 4 and gradually pushes on the syringe plunger 8 to thereby expel the feeding solution from the syringe 4 and deliver the solution to an infant. A control panel 10 is typically provided whereby the caregiver may adjust the flow rate of the pump.

The expense of syringe pumps 2 typically prevent an individual from owning such a pump, and thus requires that neonatal infants are kept in a hospital until able to feed without the assistance of a pump, or until able to tolerate the higher flow rate of a typical feeding pump. Many neonatal infants are sufficiently healthy to leave the hospital, and are only kept in the hospital because of the need for a neonatal feeding pump. Additionally, those infants that will require further enteral feeding after they have grown sufficiently will be transitioned to a more conventional enteral feeding pump—typically a peristaltic pump.

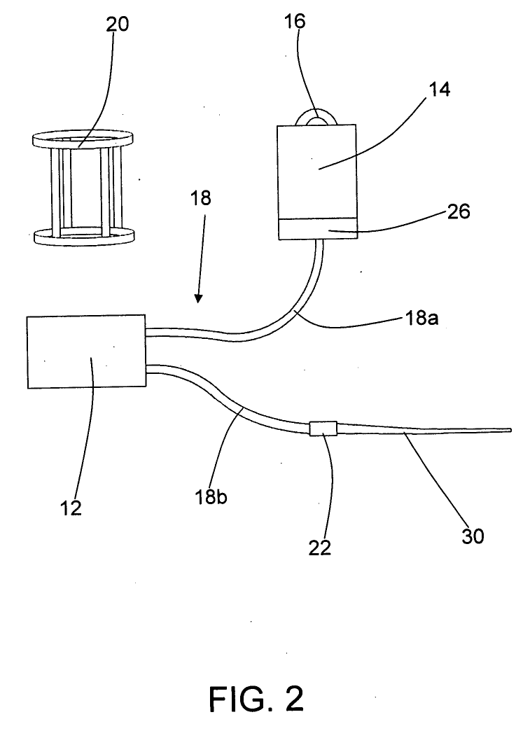

Turning now to FIG. 2, a perspective view of a feeding system according to the present invention is shown. The feeding system includes a pump 12, reservoir 14, and an infusion or feeding set 18, including a proximal portion 18a of the infusion set 18, and a distal portion 18b of feeding set 18. The reservoir 14 holds the solution to be delivered to the infant, commonly breast milk. The reservoir 14 may be a bottle or pouch, and may advantageously be the same type of container as may be connected to a breast pump. Accordingly, an adapter 26 may be provided to facilitate connection of the reservoir 14 to a proximal (or upstream) portion 18a of the feeding set 18. The feeding set 18 carries the feeding solution to the pump 12 which controls further flow of the feeding solution through the downstream or distal portion 18b of the feeding set 18.

According to a presently preferred embodiment of the present invention, the pump 12 is a peristaltic pump, which engages the feeding tube 18 non-invasively so that the distal portion 18b of the feeding set 18 carries the feeding solution from the pump 12 to an infant (not shown). The INFINITY® peristaltic pump manufactured by ZEVEX, Inc. of Salt Lake City, Utah, has been manufactured so as to be suitable for neonatal feeding, as well as for general enteral feeding. The INFINITY® pump can deliver a flow rate which is slow enough and sufficiently accurate for neonatal infants, as well as higher rates appropriate for older children and even adults. Various aspects of the INFINITY® pump and associated feeding sets are described in greater detail in U.S. Pat. Nos. 6,523,414, 6,595,950, 6,659,976, 6,685,670, 6,750,468, 6,852,094, and 6,907,788. Peristaltic pumps are advantageous as they are generally less expensive than syringe pumps. Peristaltic pumps are also easy to use and minimize the risk of contamination. The pump rotor of a peristaltic pump does not contact the feeding solution carried inside of the feeding set, eliminating the risk of contaminating the feeding solution by a poorly cleaned pump. Furthermore, peristaltic pumps are often more convenient in that they may be used in a variety of orientations, such as being mounted on a pole, attached to the side of a bed, or simply placed on a table adjacent the child.

A feeding set may be loaded into a peristaltic pump simply by wrapping the feeding set tubing around the pump rotor and securing the feeding set tubing to the pump body. Additionally, peristaltic pumps may be formed so as to work with a cartridge which carries the feeding set and is easily loaded into the pump with one hand. A cartridge typically connects the proximal portion 18a of the feeding set to an intermediate or central pumping portion of the feeding set (not shown) which is typically a piece of soft tubing such as silicone tubing, and would also connect the other end of the central pumping portion to the distal portion 18b of the feeding set 18. Thus, an feeding set may be provided which contains the proximal and distal portions of the feeding set, the pumping portion of the feeding set, a section of tubing configured for enteral feeding of an infant, and a connector or adapter configured for attaching the feeding set to a convenient feeding solution reservoir, such as a breast pump reservoir. The feeding set may also be provided with a peristaltic pump cartridge if required.

The entire feeding set may thus be provided as a sterile and disposable unit whereby an individual need only load the feeding set into the pump, connect the feeding solution reservoir, properly place the feeding tubing in the infant, and start the pump. Once the feeding has been completed, any excess feeding solution may be disposed of. The use of such an feeding set minimizes the risk of contamination of the feeding solution or improper use of the pump and feeding system.

The proximal portion 18a of the feeding set 18 may have an adapter 26 integrally formed onto or attached to an end thereof. The adapter 26 may be selected to allow a bottle, breast pump reservoir, or other convenient feeding solution reservoir to be easily attached to the feeding set 18. The distal portion 18b of the feeding set 18 may be selected so as to be an appropriate size for enteral feeding, may have a section of tubing 30 which is tapered or otherwise configured for enteral feeding, or may have a connector 22 (preferably non-IV compatible) which connects the distal portion 18b of the feeding set 18 to a section of tubing 30 configured for the enteral feeding. If the section of tubing 30 is configured for nasal feeding, it will typically be formed of an appropriate material and in an appropriate size to be sufficiently flexible for insertion into the infant's nasal passage. Additionally, coatings are available which make the tube easier to insert into the nasal passage. The selection of a particular shape, size, material, and coating for a nasal feeding tube will be known to one of skill in the art of nasal feeding or in the catheter art.

The reservoir 14 may take many forms, such as bottles or pouches. For some reservoir shapes, it is desirable to hang the reservoir 14 to ensure the proper flow of fluid and to prevent air from entering the tubing 18. A bracket or tab 16 may be provided on the reservoir 14 to allow an individual to hang the reservoir. If necessary, a cage or basket 20 may be provided which holds the reservoir 14 and allows for hanging of the reservoir. A pole or stand, such as an I.V. stand, or even the rails of a crib or bassinet may be used to hang the reservoir 14 if necessary. However, as well be discussed in additional detail below, hanging any quantity of breast milk for a prolonged period of time can be problematic because it can encourage separation of the breast milk, with the proteins, water and sugars settling to the bottom, while the milk fats rise to the top.

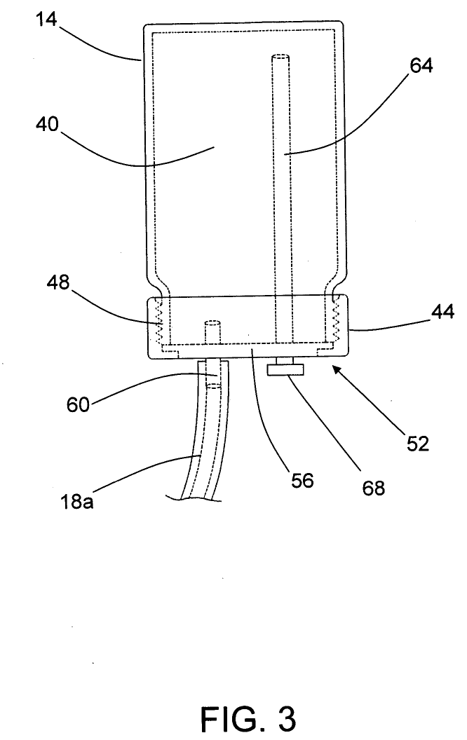

Turning now to FIG. 3, a side view of a feeding solution reservoir according to one aspect of the present invention is shown. The reservoir 14 has a bottle 40 and cap 44 which typically have threads 48 to attach the cap to the bottle (although other connection means may be used). The bottle 40 and cap 44 may be a standard baby bottle or breast pump bottle, or may have the same size threads so as to be interchangeable with these bottles. Typically, the caps 44 of baby or breast pump bottles may have an opening 52 formed therein, allowing a feeding nipple or storage insert to be inserted into the cap. Thus, an insert 56 may be provided which fits the opening 52, and which allows the reservoir 14 to be connected to the pump. The insert 56 may have a connector 60 to which the proximal portion 18a of the feeding set 18 is attached, have a hole for receiving the proximal portion 18a, or may be formed with feeding set 18 already attached. Additionally, the insert 56 may be formed with a vent 64 or be configured to receive a vent. The vent 64 prevents a vacuum from being formed as the feeding solution is drawn from the bottle 40. A filter 68 may be attached to the vent 64 which prevents liquid or solid matter from passing through the vent 64, but which allows air to enter as the feeding solution is drawn from the bottle 40. It will also be appreciated that the insert 56 may have locations wherein a person may easily pierce the insert 56 and thereby connect a vent 64 and feeding tubing 18.

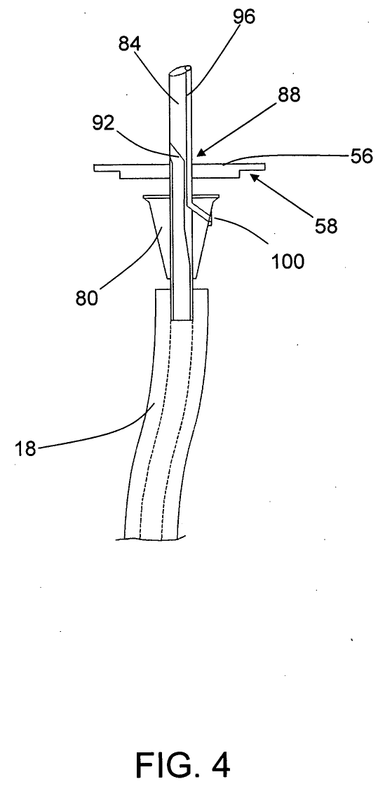

Turning now to FIG. 4, a perspective view of a reservoir insert according to the present invention is shown. The insert 56 is typically formed from a flexible material, such as rubber or silicone, and has been formed to fit with a cap and bottle such as shown in FIG. 3. A shoulder 58 is formed on the insert 56 so as to fit in the hole commonly found on the bottle caps. The insert 56 is configured to function with a vented spike type of feeding tubing connector 80. The connector 80 has a spike 84 which is used to pierce the insert 56. Accordingly, the insert 56 may have an area 88 which is easily pierced. This area 88 may be thinner, may have a hole partially formed therein, etc.

The spike 84 has a fluid carrying tube 92 and an air carrying tube 96. The fluid carrying tube 92 is connected to the proximal portion 18a of the feeding set 18, and the air carrying tube 96 is connected to an air vent 100. As liquid is drawn out of the feeding reservoir and into the feeding set 18, air is drawn into the vent 100 and through tube 96 and into the reservoir, preventing a vacuum from forming in the reservoir. The vented spike connector 80 may be part of a disposable feeding set. The insert 56 may be reusable or disposable as is desired. The insert 56 may be sufficiently inexpensive to be disposed after each use, preventing contamination of subsequent feedings from improper cleaning of the insert 56.

It will also be appreciated that the vented spike connector 80 and the insert 56 may be formed as a single piece. It may be desirable to dispose of the insert 56, connector 80, and feeding set 18 after use to prevent the contamination which could result from improper cleaning of the feeding set 18. If the feeding set 18 is designed to be disposable, it may be more cost effective to form the connector 80 and insert 56 as a single piece.

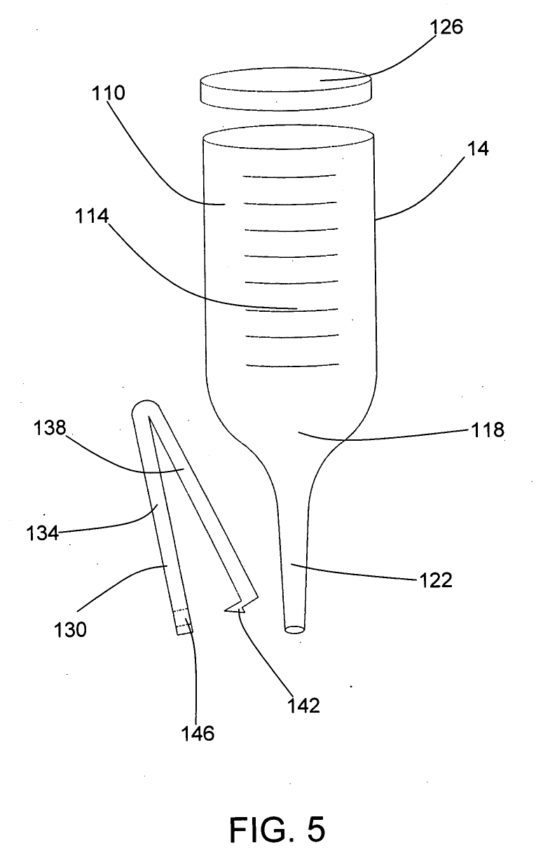

Turning now to FIG. 5, a perspective view of another reservoir according to the present invention is shown. The reservoir 14 has a cylindrical portion 110 which is sized to hold an appropriate amount of feeding solution. The cylindrical portion 110 may have markings or graduations 114 whereby a person may easily know how much liquid is in the reservoir 14. The cylindrical portion 110 is connected to a base 118 which has a tip 122 configured for attachment to the proximal portion 18a of the feeding set 18 (FIG. 1). The tip 122 may be tapered so as to be insertable into the feeding set 18, or may have a connector or appropriate shape formed therein for connection to the feeding set 18. Alternatively, reservoir 14 may be formed integrally with the proximal portion 18a of the feeding set 18 (FIG. 2) such that the tip 122 is connected to the proximal portion 18a of the feeding set 18.

The reservoir 14 may be formed so as to be generally rigid, and a cap 126 may be placed on the cylindrical portion 110 to seal the reservoir. Alternatively, the reservoir 14, and the cylindrical portion 110 in particular, may be flexible. A clip 130 may be used to close the tip 122 or the cylindrical portion 110 after filling with feeding solution so as to slectively control the size of the reservoir 14. The clip 130 may have a base 134 and an arm 138 which close together, and a latch 142 which engages a portion of the base 130, such as hole 146, to hold the clip closed. The cylindrical portion 10 of the reservoir 14 would preferably be sufficiently flexible that it is easily folded flat and placed in the clip 130, and that the cylindrical portion 110 is flattened or collapsed as the feeding solution is drawn from the reservoir 14, preventing a vacuum from forming inside of the reservoir. The clip 130 may be placed on the reservoir 14 so as to eliminate air from the reservoir, allowing the reservoir to operate in many positions independent of a hanging pole, etc., without introducing air into the proximal tubing 18a.

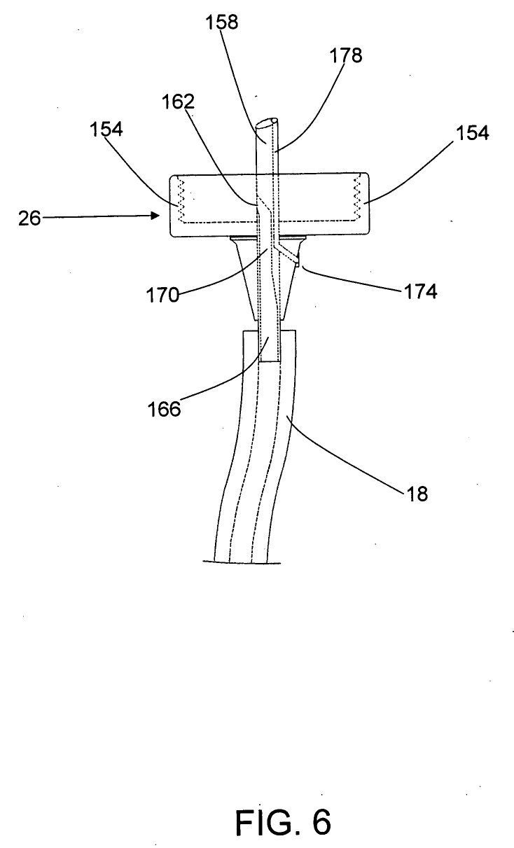

Turning now to FIG. 6, a side view of an adapter according to the present invention is shown. The adapter 26 is configured for attaching a feeding reservoir to an feeding set 18 to thereby feed a neonatal infant. The adapter 26 is configured for use with a baby bottle or breast pump bottle and is accordingly provided with threads 154 configured for attachment to such a bottle. The adapter 26 serves as a cap for the bottle. A spike 158 is formed with a fluid outlet 162 which is connected to a tip 166 via channel 170. Feeding solution flows into the opening 162, through the channel 170, through tip 166, and into the feeding set 18 as the pump 12 (FIG. 2) draws fluid from the reservoir. An air opening 174 and channel 178 allow air to flow into the reservoir to prevent the formation of a vacuum as the feeding solution is withdrawn from the reservoir.

Those skilled in the art will appreciate that it may be desirable to use a relatively small bottle with the adaptor 26, i.e. a few milliliters, so that the solution in the bottle does not hang for a prolonged period of time. The longer the solution hangs, the more likely it is that there will be separation between the milk fats and the proteins and sugars. This can result in the neonatal infant getting inconsistent amounts of nutrients, where it is generally desirable for the feeding solution to be relatively consistent. Once the child is able to eat in larger volumes or at faster flow rates, a similar adaptor 26 can be used with a larger bottle, as the larger volume of breast milk will have less time in which to separate.

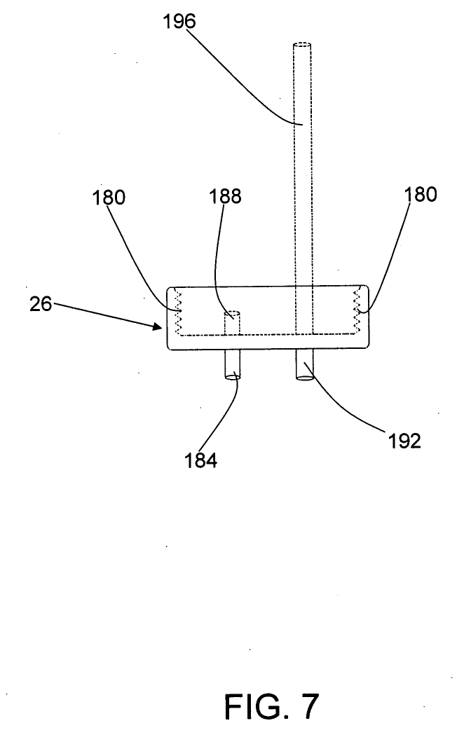

Turning now to FIG. 7, a side view of an adapter according to the present invention is shown. The adapter 26 is designed to replace the cap of a baby bottle or breast pump bottle, and accordingly is formed with threads 180 configured for engaging such a bottle. As noted above, the bottle can be configured to contain any desired volume of feeding solution.

The adapter has a first connector 184 through which a first channel 188 passes, and a second connector 192 through which a second channel 196 passes. The adapter 26 may be configured such that the feeding set 18 is connected to the first connector 184 and draws liquid through the first channel 188 with the bottle in an inverted position. Air is then allowed to enter the bottle through channel 196 to prevent the formation of a vacuum. Alternatively, the adapter 26 could be configured such that the feeding set 18 is connected to the second connector 192 such that the pump draws feeding solution from the second channel 196 with the bottle in an upright position, allowing air to enter through the first channel 188.

It may be advantageous to provide an adapter 26 which allows for the bottle of feeding solution to be maintained in an upright or inverted position as is necessary for a particular application. It is further advantageous to provide a simple to use and inexpensive cap to allow a conventional baby bottle or breast pump bottle to be used as a neonatal feeding reservoir. This allows the feeding solution, typically milk, to be easily provided to the infant with a minimal risk of contamination. The adapter 26 may also be produced for a low cost, allowing the adapter 26 to be discarded after each use, reducing the risk of contamination of the feeding solution. For safety reasons, the adapter should be configured to only attach to other enteral feeding connections and not compatible with parenteral IV luer adapters.

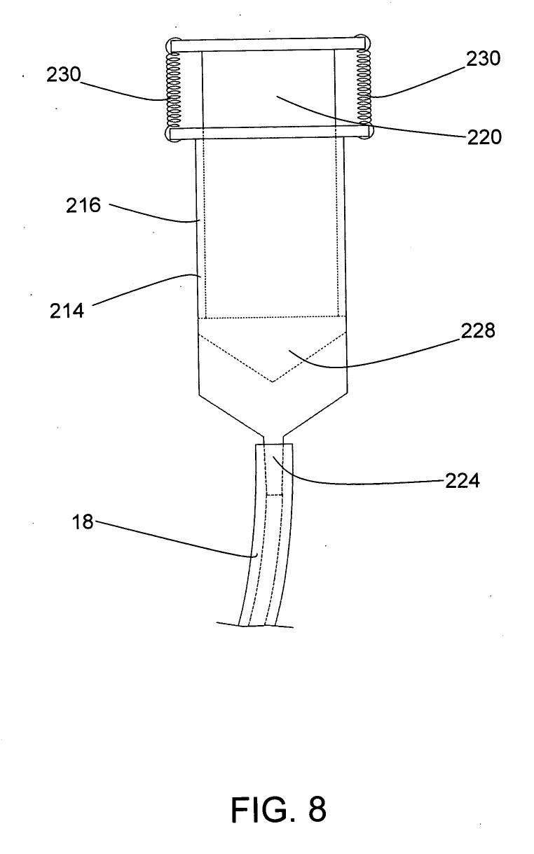

Turning now to FIG. 8, a perspective view of another reservoir according to the present invention is shown. The reservoir 214 is formed from a syringe, having a syringe body 216 and plunger 220. The syringe body 216 may be selected to have an elongate tip 224 to which the feeding set 18 may be attached. Alternatively, the syringe body 216 may have a specialized adapter formed thereon for receiving the feeding set 18 or an intermediate connector (not shown). By withdrawing the plunger 220 and piston 228 from the syringe body 216, an individual may draw milk or another feeding solution into the syringe. As the pump 12 (FIG. 1) draws milk out of the reservoir 214, the syringe plunger 220 may move unassisted back into the syringe body 216. Most syringes, however, are sufficiently resistant to movement that an enteral feeding pump cannot generate a sufficient vacuum to draw the plunger 220 toward the tip 224. This creates an undesirable vacuum within the syringe. To remedy such a situation, one or more mechanical biasing elements or members 230 may be used to urge the plunger 220 into the body 216.

The biasing member may be a spring or elastomeric material such as rubber. The reservoir 214 shown would allow a user to avoid the introduction of air bubbles into the solution and avoid the formation of a vacuum within the reservoir 214 while dispensing the feeding solution. While requiring the transferring of the feeding solution into the syringe, the use of a syringe of a reservoir is advantageous as syringes are readily available and disposable, and provides a reservoir which does not require a vent to eliminate the formation of a vacuum within the reservoir. Additionally, the configuration of the reservoir 214 allows it to be used regardless of orientation. Thus, the syringe can be laid on its side where there will be less separation of breast milk contained therein. Additionally, even if there is separation, the variance in the solution delivered will be lower, as the piston 220 would tend to force both milk fats (at the top) and proteins/sugars (at the bottom) into the tube 18 simultaneously. If the syringe is held upright as shown in FIG. 8, watery solution with protein and sugars would tend to be fed to the neonatal infant first, while the richer milk fats would be provided last.

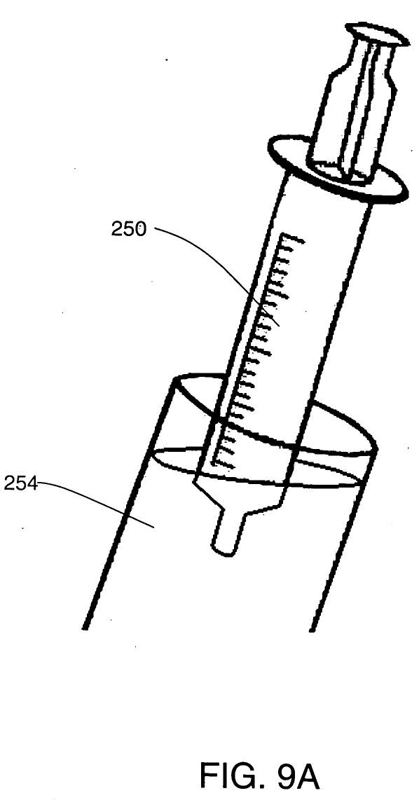

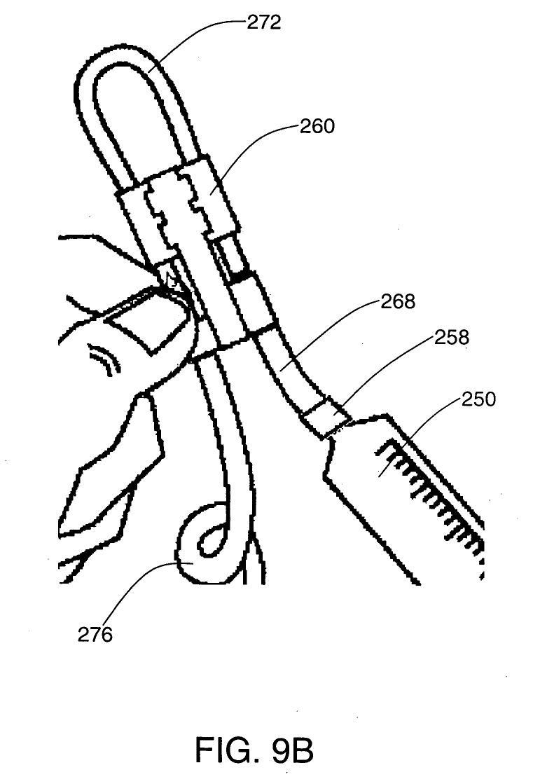

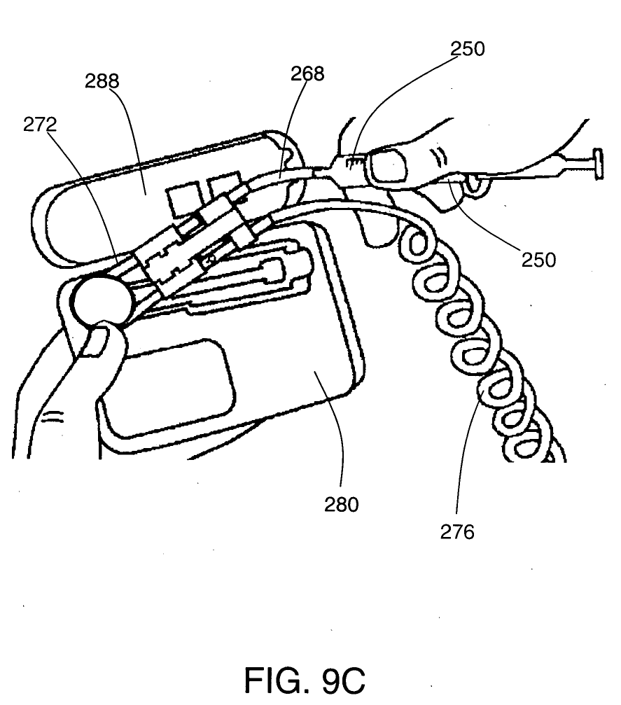

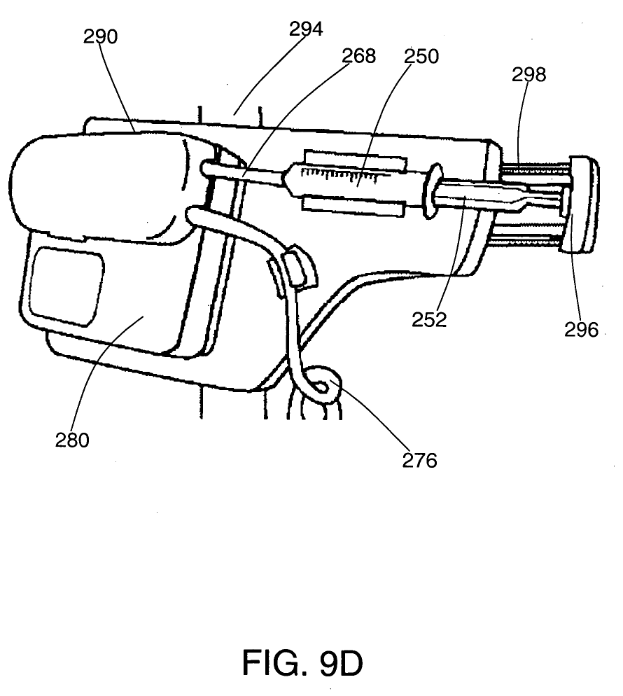

Turning now to FIG. 9A, there is shown a side view of a syringe 250 being used to draw breast milk from a container 254. As was mentioned previously, many neonatal infants are only able to eat in very small quantities. Thus it is common for the mother to use a breast pump and provide an amount of breast milk which will cover multiple feedings. The unused breast milk is kept in a refrigerator to minimize bacterial growth. One advantage of using the syringe 250 is that it enables the user to easily determine the amount of breast milk which has been withdrawn. Thus, it is easy to record the volume of milk being delivered to the neonatal infant.

Turning now to FIG. 9B, there is shown a side view of the syringe 250 being connected to the cassette 260 of an enteral feeding set 264. The cassette includes an inlet (upstream) tubing 268, an intermediate portion 272 configured for engaging the rotors of the feeding pump, and outflow (downstream) tubing 276 for delivering the feeding solution to the neonatal infant. As shown, the syringe 250 may be connected to an adapter or luer 258 which is attached to or formed as part of the inlet tubing 268.

As shown in FIG. 9B, the outflow tubing 276 is preferably a coiled microbore or minibore tubing. The coil allows the tubing 276 to stretch to the patient, but keeps it from becoming tangled in other tubing and wires which may be used to monitor the patient, provide I.V. drug administration, etc. Preferably, the tubing is constructed from a material which maintains a mechanical memory to ensure a kink free, non-restrictive flow of breast milk. The microbore or minibore also allows very finite amounts of breast milk or other feeding solutions to held or contained prior to delivery to the neonatal infant. Thus, it is preferable that the tubing 276 also has a small bore extending therethrough. The small inner diameter of the microbore tubing also aids in minimizing the separation of the fats from the watery portion of the breast milk.

Turning now to FIG. 9C there is shown a perspective view of the cassette 260 being loaded into an enteral feeding pump 280. The intermediate portion (or pump rotor tubing segment) 272 is wrapped around the rotor 284 of the enteral feeding pump and the cassette 260 nested in place. A door 288 of the pump 280 is then shut to hold the cassette in place.

FIG. 9D shows the enteral feeding pump 280, and the syringe 250 disposed on a mount 290. The mount 290 may be configured to receive the pump 280 and hold it to a support, such as an I.V. pole 294, or may be configured to simply attach to the pump 280. The mount 290 is also preferably configured to hold the syringe 250 and preferably includes a biasing member 298 which applies force to the piston 252 of the syringe. Those skilled in the art will appreciate that the biasing member 296 can use springs or elastomeric elements 298 to apply pressure to the piston sufficient that the suction provided by the enteral feeding pump will draw the breast milk or other feeding solution from the syringe 250, through the inlet tubing 268, and pump it through the downstream tubing 276 to the patient.

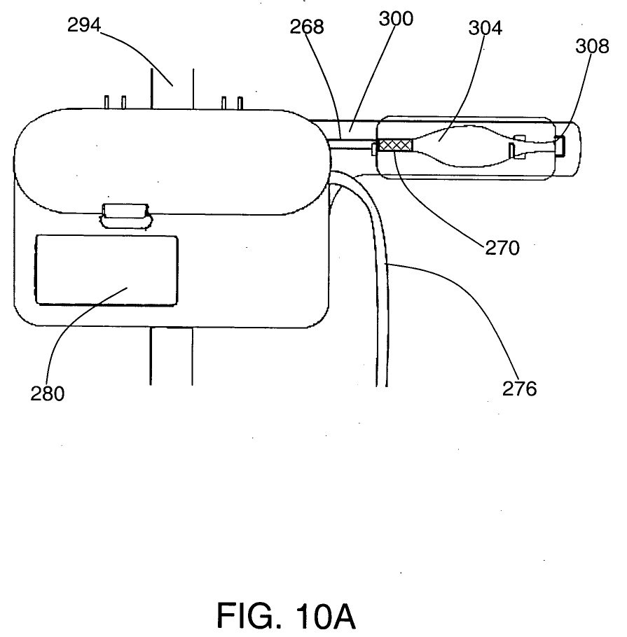

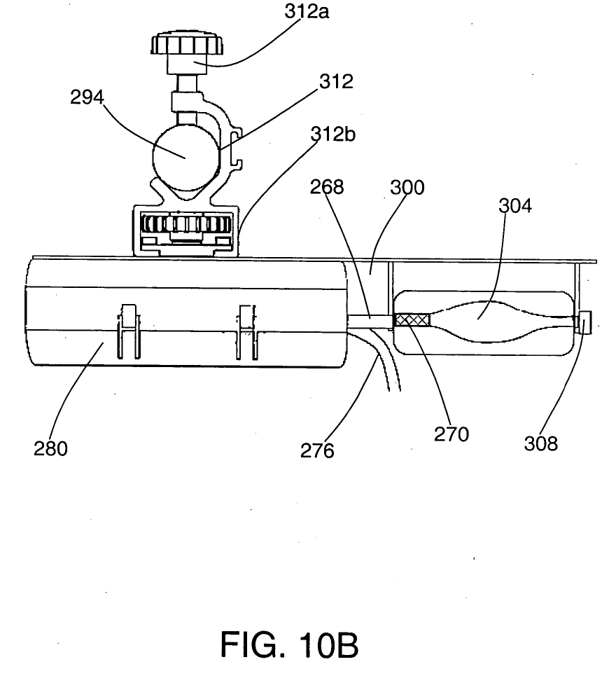

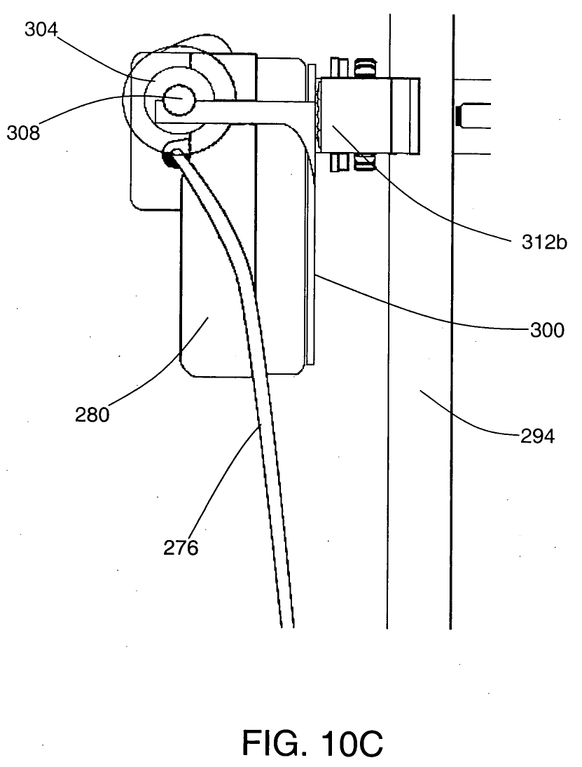

Turning now to FIG. 10A, there is shown an alternate embodiment of the invention. The enteral feeding pump 280 is attached to an IV pole 294. If desired, the pump 280 can be attached to a bed rail, or simply disposed on any suitable surface.

Attached to the enteral feeding pump 280 is a mount 300. Rather than a syringe, the inlet tubing 268 of the feeding set is attached to a reservoir in the form of a radially expandable container 304. The container 304 is preferably made from an elastameric material. Being elastomeric in construction, the container 304 will act as its own infusion device creating a positive interal pressure when filled with breast milk. This will aid in delivering the breast milk to the pump, thus minimizing fluid separation and eliminating a partial vacuum.

Disposed on the container 304 is an injection port 308 having a one-way valve. This allows a user to inject the milk into the container 304. The milk is then drawn out of the container, through the inlet tubing 268 and out to the patient through the downstream tubing 276. One advantage of the container 304 is that it can hold small quantities of milk (anywhere from 5 mL to 150 mL depending on intended use). Additionally, because contents of the container 304 extend generally horizontally, any separation between milk proteins and sugars and the milk fats is minimized. As the milk is withdrawn laterally from the container, a more even blend of fats, milk proteins, and sugars is delivered to the infant. A static mixer 270 can also be included. As will be explained in additional detail below, a static mixer 270 helps to minimize separation of the milk fats from the water and proteins.

FIGS. 10B and 10C show a top view and a side view, respectively, of the pump 280, inlet and outlet tubing 268, 276 and the mount 300 and container 304. The pump 280 and mount 300 are held to an IV pole 294 by a clamp 312. Such clamps are well known in the enteral feeding arts. The clamp 312 includes a first end 312a configured for engaging the pole 294 and a second end 312b configured for engaging the pump 280 and/or mount 300. Ideally, the second end 312b is configured to allow selective positioning of the pump 280 and/or mount 300.

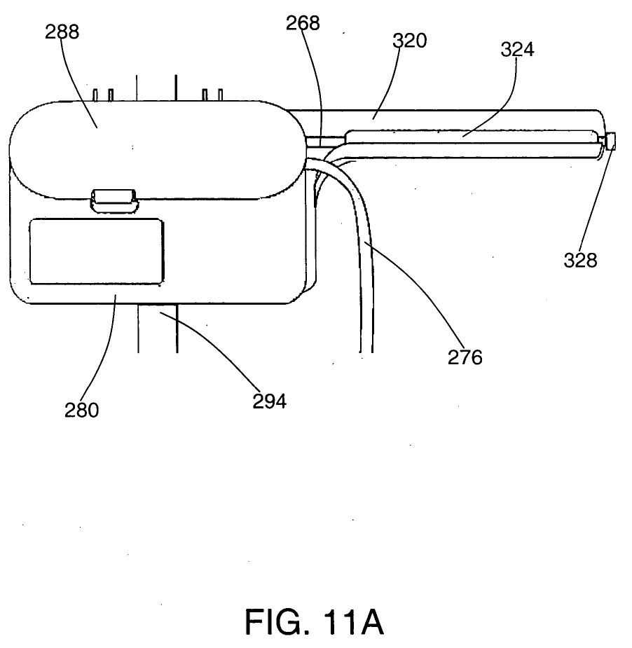

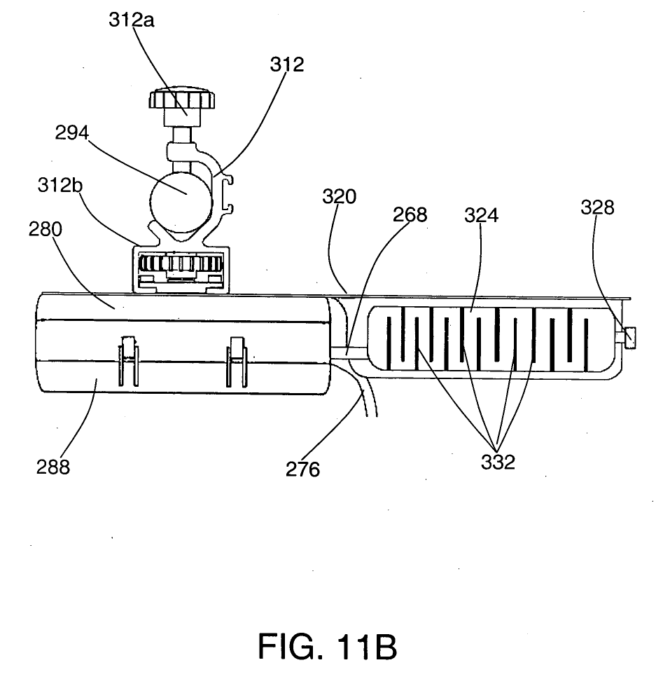

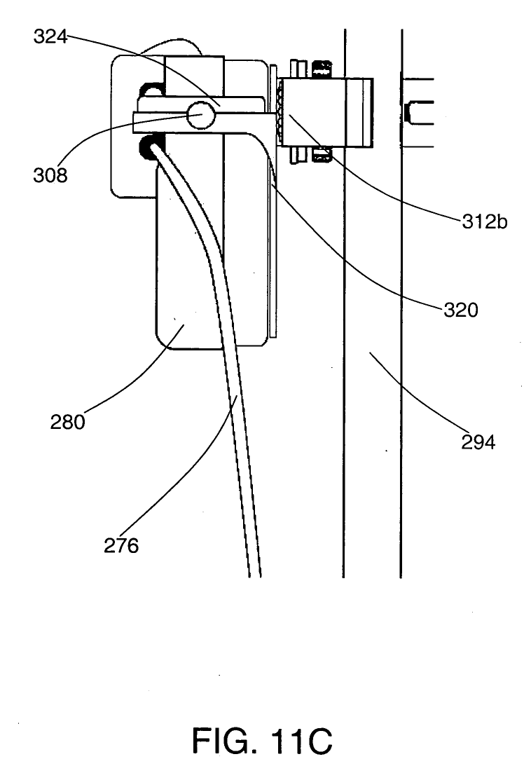

Turning now to FIGS. 11A through 11C, there are shown front, top and end views, respectively, of the pump 280 attached to a pole 294. Rather than the container 304, a different feeding solution storage system is used. Attached to the pump 280 is a mount 320 which supports a container or reservoir 324 attached to the inlet tubing 268. The reservoir 324 has a port 328 through which breast milk or other feeding solutions can be injected into the container. Additionally, the reservoir 324 may be collapsible, or may be vented to prevent the buildup of a vacuum within a container.

As noted above, one concern which is present with the administration of breast milk to a neonatal infant is that the breast milk will tend to separate if it is allowed to sit for too long. To limit the amount of separation, the reservoir 324 is configured so that it extends generally horizontally (i.e. the volume of milk contained therein is more horizontal than vertical). Additionally, a plurality of walls 332 can be disposed inside of the reservoir. Because of the relatively low height, the distance between any rising milk fats and sinking water containing proteins and sugars is kept fairly small. Additionally, the walls 332 encourage movement of all of the milk as it is being pumped to the neonatal infant, rather than simply milk immediately adjacent the inlet tubing 268. To the extent that there is separation, the lateral movement of the milk out of the reservoir 324 tends to draw a blend of fats and water contained in the breast milk. If a container were simply hung in a conventional manner, the proteins and sugars would be delivered first, followed by milk fats which have risen to the top of the solution.

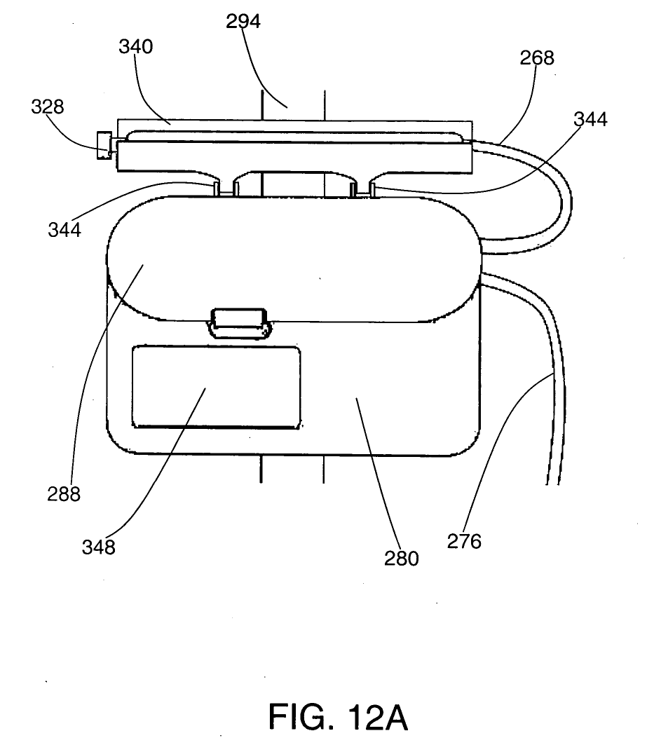

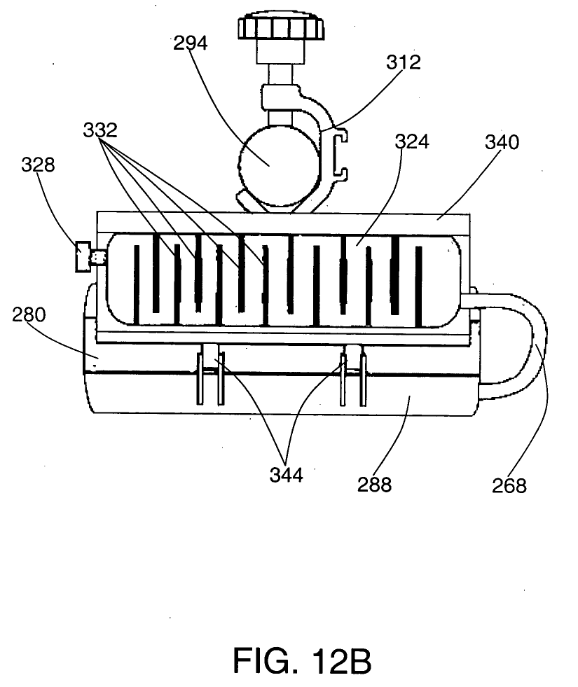

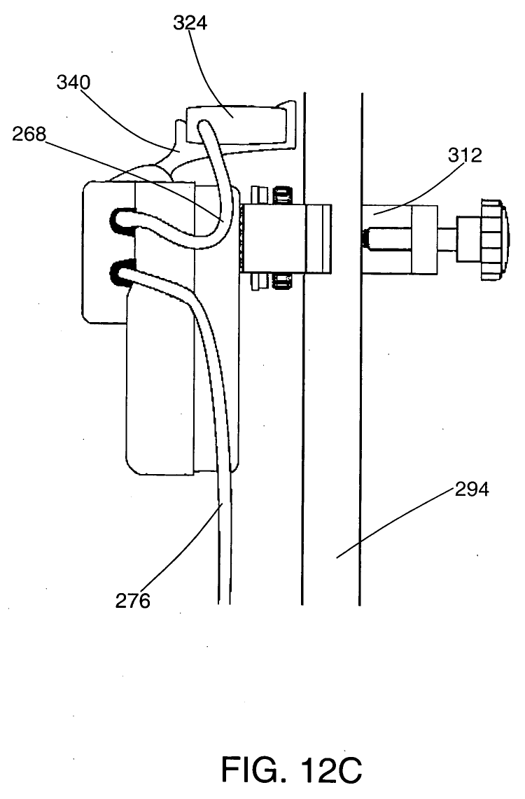

Turning now to FIGS. 12A through 12C, there are shown front, top and end views of an alternate configuration of an embodiment of the invention. The enteral feeding pump 280 is attached to the IV pole 294. While shown attached with a clamp 312, it will be appreciated that the enteral feeding pump 280 can be attached with conventional attachment means, attached to a bed frame or simply placed on a table.

A mount 340 is attached to the enteral feeding pump 280. Rather than being attached to the back side, as shown previously, the mount 340 is attached to the hinges 344 which enable opening and closing of the door 288. Disposed on the mount 340 is a reservoir, such as the reservoir 324. The position of the reservoir 324 above the pump provides easy access to the port 328 for injecting feeding solution. It also allows a health care worker to quickly view both the contents of the reservoir 324 and information provided by a screen 348 of the enteral feeding pump, such as the volume dispensed to the patient, the volumetric rate of delivery, etc.

Ideally, the mount 340 is configured so as to be sloped slightly toward the end where the reservoir 324 is connected to or formed integrally with the inlet tubing 268. This encourages the breast milk or other feeding solution to work its way out of the reservoir 324 and into the tubing of the feeding set. Additionally, one advantage of having the mount 340 attached to the hinges 344 of the pump 280, is that this allows reservoir 324 to be adjusted or to be rocked back and forth to minimize any separation. It also allows the milk to be disposed at an angle which is most convenient for viewing by medical personnel. It will be appreciated that the reservoir 324 can be collapsible or may include a vent to prevent vacuum pressure from interfering with solution delivery by the pump 280.

While shown in conjunction with the ZEVEX ENTERALITE INFINITY enteral feeding pump, it will be appreciated that a variety of different pumps may be used in conjunction with various aspects of the present invention. One advantage of the present invention, however, is that it facilitates the use of less expensive enteral feeding pumps, while maintaining safety and control over the quantity and quality of the breast milk or other feeding solution which is being administered to the neonatal infant.

Turning now to FIG. 13, there is shown a side view of a reservoir 360 made in accordance with the principles of the present invention. The reservoir 360 has a port 364 for injecting breast milk or other feeding solution. In the alternative, the reservoir can have any variety of openings to allow the milk to be placed therein. Inside the reservoir 360 are a plurality of walls 368. These walls 368 may be formed by radio frequency welding of the two sheets of thin walled plastic, as is customary in the art for disposable bag manufacturing. As the breast milk or other feeding solution is removed from the reservoir 360, the milk flows around the walls. If the milk is allowed to hang for a prolonged period of time, the ability of the milk to separate is limited by the walls. Instead of the milk fat layer rising to the top of the container while the proteins and sugars sink to the bottom, the separation will tend to happen between adjacent pairs of walls. Thus, the distance between the top of the milk fat layer and the bottom of the watery proteins and sugars will be little more than the spacing of the walls. Thus, the milk delivered through the outlet port 372 to a neonatal infant will be more consistent.

Turning now to FIG. 14, there is shown a front view of an enteral feeding device similar to that shown in FIG. 12A. Added to the mount 340 adjacent the hinges 344 is a motor unit 380. The motor unit 380 may be powered by a electricity supply line 384, may be powered by the pump 280, or may be mechanically driven, such as by a winding mechanism and a spring. The function of the motor unit 380 is to cause the mount 340 to pivot with respect to the pump 280 to thereby shake the contents of the container 324. This in turn, minimizes separation within the feeding solution, or between the mixture of feeding solution and medication, etc.

As was noted above, the separation of milk fats from the water and protein is generally not desirable. Additionally, the separation of medicine from the breast milk or other feeding solution is also undesirable. In some instances, a child's parent or the patient must wake periodically (as often as every hour or two) to shake a container of feeding solution to prevent a significantly uneven dose of medication being delivered to the patient. By providing a motor unit 380, the solution can be moved to thereby minimize separation and minimize handling by the patient, parent, etc.

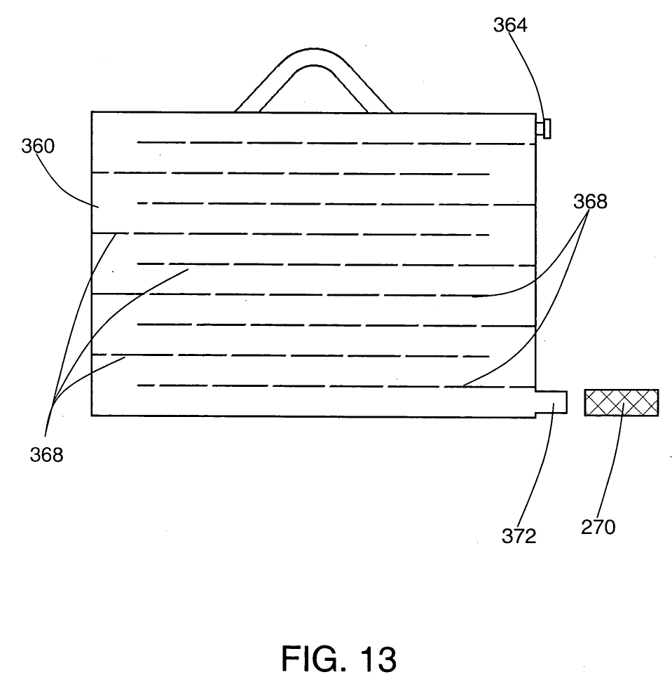

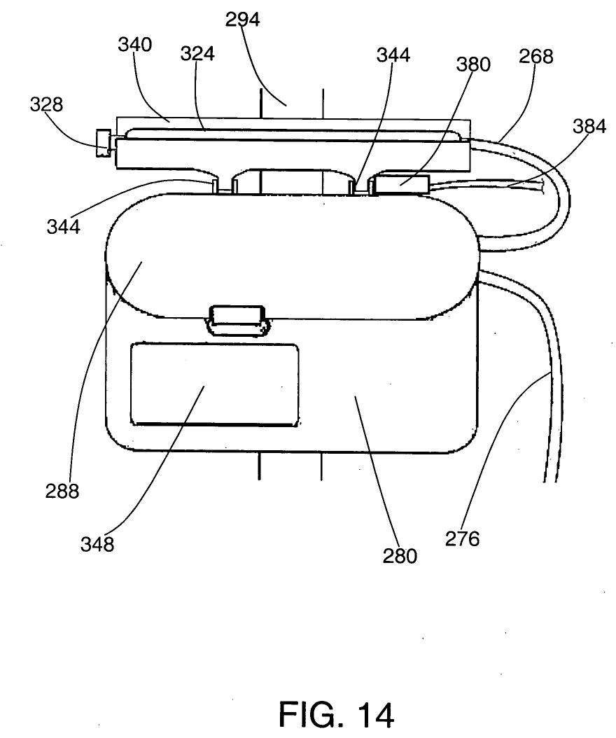

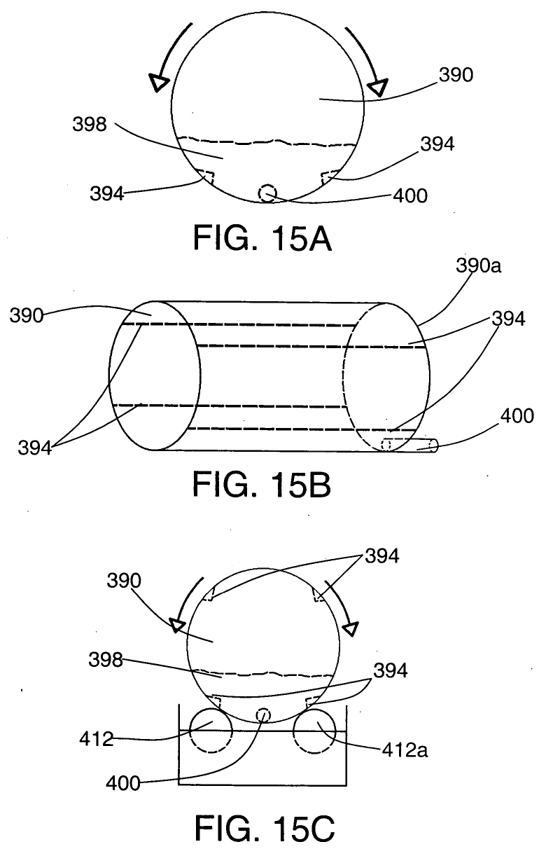

FIG. 15A shows a cross-sectional view of an alternate configuration of a solution container 390, designed to minimize separation within the enteral feeding solution prior to delivery to the patient. The container 390 includes a pair of protrusions 394 which act as mixing blades for a solution 398 disposed within the container. The protrusions 394 typically extend the length of the container 390. As the container 390 rotates, the protrusions 394 mix the solution inside, thereby minimizing separation. The container 390 may be rotated completely, or may simply be rotated a set amount, i.e. 15-25 degrees, and then rotated in the opposite direction. Either way, the container 390 will minimize the separation within the feeding solution 398. An outlet port 400 allows the solution 398 to be withdrawn from the container 390.

FIG. 15B shows a perspective view of the container 390. Disposed at one end 390a of the container 390 is an outlet port side 400 through which the feeding solution is passed to an enteral feeding set. The container 390 shows four protrusions 394, but any number of projections may be used as is necessary.

FIG. 15C shows an end view of the solution container 390 disposed on a rocker motor 404. The rocker motor 404 includes a housing 408 and a pair of supports 412 which are preferably elongate rotatable cylinders. One support 412a is preferably connected to a motor (either electric or mechanical) which rotates the support to thereby cause rotation of the container 390. The rotation may be unidirectional, or may cause the container 390 to rotate back and forth some desired amount, i.e. between 15-25 degrees. Either way, the solution in the container 390 is mixed and separation is minimized.

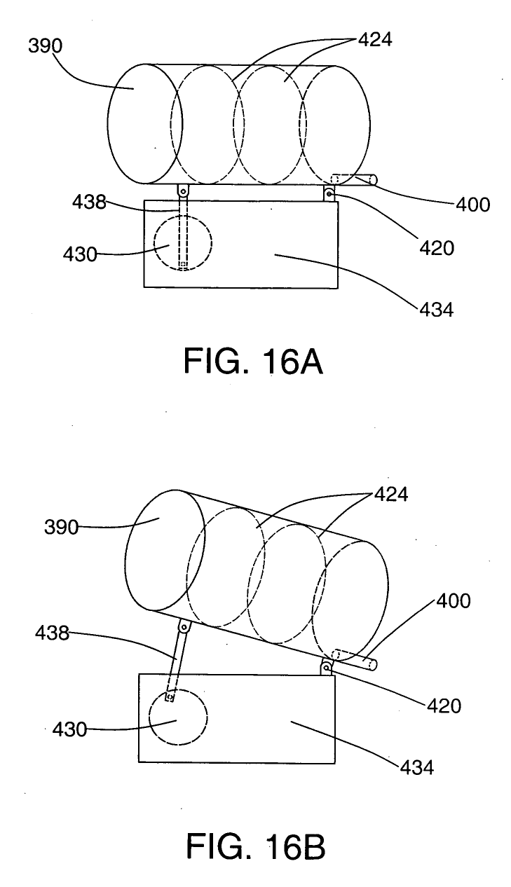

FIG. 16A shows a perspective view of an alternate configuration of a solution container 390 designed minimize separation within the feeding solution. The container 390 is shown with a plurality of baffles 424 for encouraging mixing. However, one skilled in the art will appreciate that baffles 424 are not required and that other structures could work as well. The container 390 is connected to the rotor 430 of a mixing mechanism 434 by a drive line 438. The rotor of a peristaltic pump may be used as the mixing rotor 430. As shown in FIG. 16B, as the rotor 430 of the mixing mechanism 434 rotates, it acts on the drive line 438 to raise and lower the container 390, thereby disturbing the contents of the container and causing the feeding solution, etc., to mix. It will be appreciated that the drive line 438 cause be modified to pivot a mount, such as that discussed above, or otherwise modified to agitate the container and cause mixing of the solution. A hinge may be used to pivotably attach the container 390 to the mixing mechanism 434. It will also be appreciated that the container 390 will include a outlet port 400, and it will be desirable to maintain the liquid around the outlet port so that air is not delivered to a patient.

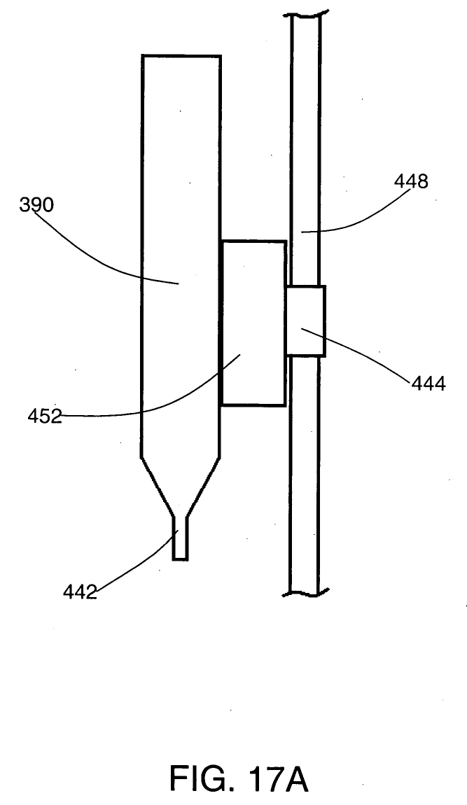

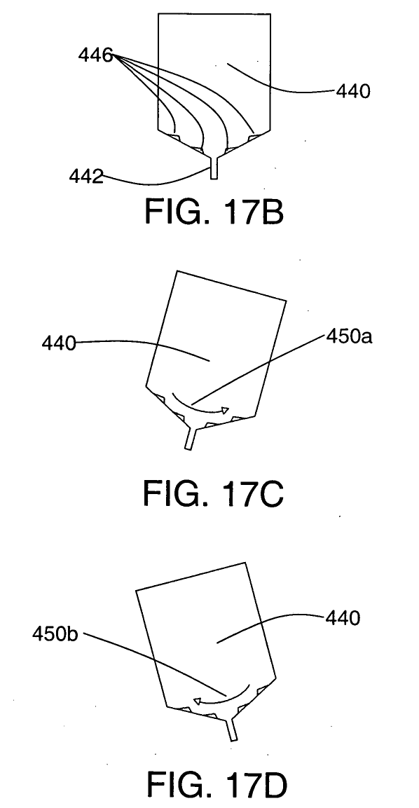

Turning now to FIG. 17A, there is shown a side view of a solution container 390 mounted by a clamp 444 to a pole 448. The clamp 444 can include a motor unit 452 or other mechanism to cause the container 390 to pivot back and forth as shown in FIGS. 17B, 17C and 17D. Thus the contents of the container 390 are periodically or continually shaken to minimize separation. An outlet port 442 is provided at the bottom of the container 390 to allow the liquid to flow out of the container and to a patient. This reduces the possibility of milk fats significantly separating from water and proteins, as well as minimizing the separation of the medication from the feeding solution. Thus, this aspect of the invention has applicability both to infants and to enteral feeding pump users of all ages who are concerned about fluid separation. FIG. 17B also shows protrusions 446 which may be formed along the bottom and/or sides of the container 390 to aid in the mixing of the liquid.

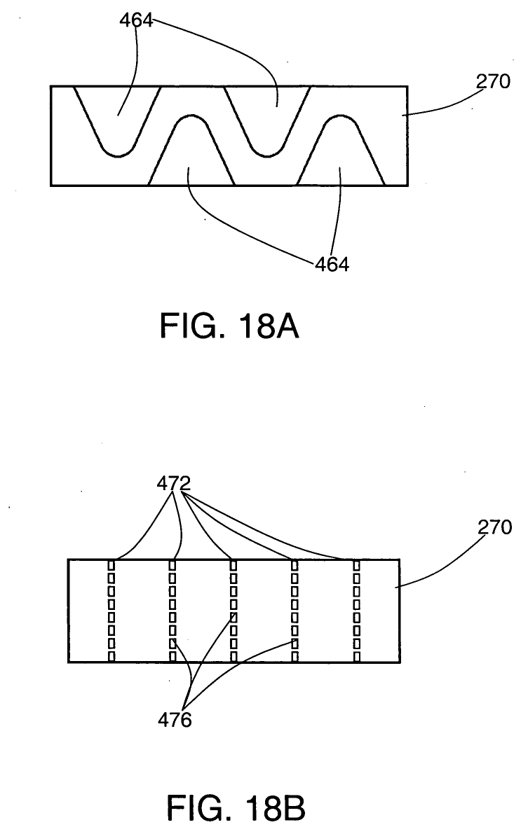

FIG. 18A shows a close-up view of a static mixer 270. The static mixer 270 can include a plurality of baffles 464 or other structures which agitate or otherwise mix the solution as it passes therethrough. Thus, in the present application applicant uses the term static mixer to refer to a mixer which utilizes one or more static elements to mix the fluid as the fluid flows through the mixer and past the static elements. FIG. 18B shows another static mixer 270 which includes one or more plates 472 having holes 476 formed therethrough. The static mixer is typically designed to provide a broken or tortuous path for the liquid to thereby cause mixing of the liquid as flow occurs. This causes the mixture, be it feeding solution or feeding solution and medication, to mix, thereby reducing separation. It will be appreciated that a combination of protrusions, baffles, plates, etc. may be used to create a desired flow path which mixes the liquid flowing therethrough. Those skilled in the art will appreciate that the design of the baffles or other structures to cause mixing can be selected to be particularly effective with solutions of the consistency of enteral feeding solutions. The static mixers described with reference to FIGS. 18A and 18B may be used in combination with any of the above embodiments of the invention.

There is thus disclosed an improved methods and apparatuses for controlled feeding of an infant. It will be appreciated that numerous changes may be made to the various embodiments of the present invention without departing from the scope of the claims. The appended claims are intended to cover such modifications.

What is claimed is:

1. A method of feeding a neonatal infant comprising:
selecting a baby bottle being filled with feeding solution;
connecting the baby bottle to a feeding pump;
operating the pump to thereby deliver the feeding solution to an infant.

2. The method of claim 1, wherein the infant is fed nasally.

3. The method of claim 1, wherein the infant is fed enteraly.

4. The method of claim 1, wherein the method further comprises attaching an adapter to the bottle and attaching the adapter to an feeding set.

5. The method of claim 1, wherein the baby bottle is a breast pump bottle.

6. The method of claim 1, wherein the pump is a peristaltic pump.

7. An adapter comprising a surface configured for covering the opening of an infant bottle, and configured for placement in the opening of an infant bottle cap, the adapter further comprising a vent and means for connecting the adapter to the inlet tubing of a feeding pump.

8. The adapter of claim 7, wherein the vent is configured for allowing air to enter then bottle and for preventing the passage of liquid or solid material through the vent.

9. The adapter of claim 7, wherein the means for connecting the adapter to the inlet tubing of a feeding pump and the vent are disposed inside of the circular shoulder.

10. The adapter of claim 7, wherein the means for connecting the adapter to the inlet tubing of an feeding pump comprises a cylindrical connector.

11. The adapter of claim 7, wherein the means for connecting the adapter to the inlet tubing of an feeding pump comprises a pierce-able location configured for receiving a tubing.

12. The adapter of claim 7, wherein the means for connecting the adapter to the inlet tubing of an feeding pump comprises a pierce-able location and further comprising a vented spike configured for placement through the pierce-able location, the vented spike having a conduit configured for carrying liquid from the bottle to an feeding tubing and a conduit configured for carrying air into the bottle to thereby prevent the formation of a vacuum.

13. A reservoir for holding feeding solution for delivery to an infant comprising:
a first end configured for attachment to the inlet tubing of a feeding pump;
a middle portion configured for receiving a feeding solution; and
a second end configured to allow placement of feeding solution into the reservoir; and
wherein at least the middle portion and the second end are formed from a flexible material.

14. The reservoir of claim 13, further comprising a clip configured for holding the second end of the reservoir closed after feeding solution is placed in the reservoir.

15. A system for holding feeding solution for delivery to an infant comprising:
an elongate reservoir having an open first end, a second end opposite the first end and configured for attachment to the inlet tubing of a feeding pump, and an interior surface extending between the first end and second end;
a plunger configured for slide-able engagement with the interior surface of the reservoir so as to seal against the interior surface; and
a biasing member configured to bias the plunger towards the second end of the reservoir.

16. A method for feeding an infant comprising:
selecting a reservoir having a feeding solution disposed therein, the reservoir selected from the group consisting of a baby bottle and a breast pump milk reservoir;
attaching an adapter configured for attaching then reservoir to an feeding set to the reservoir opening;
attaching an feeding set to the adapter;
delivering the feeding solution to an infant.

17. The method of claim 16, wherein the adapter further comprises a vent configured for allowing air to enter the reservoir as the feeding solution is withdrawn from the reservoir.

18. A method for feeding a neonatal infant comprising:
selecting a collapsible reservoir;
filling the collapsible reservoir with feeding solution;
attaching the collapsible reservoir to an feeding set; and
delivering the feeding solution to an infant.

19. The method of claim 18, wherein the collapsible reservoir is formed from flexible plastic.

20. The method of claim 18, wherein the collapsible reservoir comprises a syringe.

21. The method of claim 18, wherein a peristaltic pump is used to deliver the feeding solution to the infant.

22. A method of feeding a neonatal infant comprising:
filling a reservoir feeding solution;
attaching the reservoir to a feeding set;
loading the feeding set into a peristaltic pump; and
delivering the feeding solution to an infant.

23. The method of claim 22, wherein the method comprises selecting a reservoir that is a breast pump bottle.

24. The method of claim 22, wherein the method comprises using a reservoir in the form of a syringe.

25. The method of claim 24, wherein the syringe comprises a plunger and wherein the method comprises disposing a biasing member in communication with the syringe to apply pressure to the plunger.

26. The method of claim 25, wherein the flow of solution through the feeding set is controlled by the enteral feeding pump.

27. The method of claim 22, wherein the method comprises selecting a reservoir formed by a radially expandable container.

28. The method of claim 27, wherein the further comprises injecting the solution into the radially expandable container through a port disposed in communication therewith.

29. The method of claim 28, wherein the method comprises selecting a reservoir having a plurality of walls formed therein for directing flow of the solution within the reservoir along a tortuous path.

30. A system for feeding a neonatal infant comprising:
a peristaltic pump;
a reservoir configured for holding feeding solution;
an feeding set having a proximal portion configured for attachment to the reservoir, a pumping section configured for insertion into the peristaltic pump, and a distal portion configured for delivering a feeding solution to an infant.

31. The system of claim 30, wherein the reservoir comprises a bottle.

32. The system of claim 30, wherein a distal end of the distal portion of the feeding set is tapered to a smaller diameter than the proximal end of the distal portion.

33. The system of claim 30, wherein the distal end of the distal portion has a microbore.

34. The system of claim 30, further comprising an adaptor for connecting the feeding set to the reservoir.

35. The system of claim 34, wherein the reservoir comprises a bottle and wherein the adaptor comprises a cover for the bottle and a means for piercing the cover.

36. The system of claim 30, wherein the reservoir further comprises a port for injecting feeding solution into the reservoir.

37. The system of claim 30, wherein the reservoir comprises a radially expandible container.

38. The system of claim 30, wherein the reservoir comprises an elongate reservoir disposed generally horizontally.

39. The system of claim 30, wherein the reservoir comprises an inlet, and outlet and a plurality of walls disposed therein for directing fluid flow between the inlet and outlet.

40. The system of claim 30, wherein the plurality of walls are disposed generally parallel to one another.

41. The system of claim 30, further comprising a mount for holding the reservoir.

42. The system of claim 40, wherein the mount is pivotably attached to the enteral feeding pump.

43. The system of claim 30, wherein the reservoir comprises a syringe.

44. The system of claim 42, wherein the syringe comprises a plunger and further comprising a biasing member disposed in communication with the plunger so as to apply force to the plunger.

45. The system of claim 30, wherein the reservoir is configured to hold less than 5 ml of feeding soultion.

46. A reservoir for delivering feeding solution to a neonatal infant, the reservoir comprising a first port for injecting feeding solution into the reservoir and a second port for releasing feeding solution into tubing of an feeding set, the reservoir comprising a plurality of generally parallel walls disposed therein for directing fluid flow from the first port to the second port in generally horizontal segments.

47. A system for delivering feeding solution comprising:
a reservoir for holding the feeding solution, comprising an outlet port for allowing withdrawal of the feeding solution; and
means for agitating the solution in the reservoir to thereby inhibit separation of the feeding solution.

48. The system of claim 46, wherein the reservoir is cylindrical and wherein the means for agitating the solution comprises means for rolling the reservoir.

49. The system of claim 46, wherein the reservoir comprises a projection disposed in contact with the feeding solution.

50. The system of claim 48, wherein the projection comprises an elongate rib.

51. The system of claim 48, wherein the projection comprises an annular rib.

52. The system of claim 46, wherein the means for agitating the solution pivots the reservoir alternately between a first position and a second position.

53. A system for delivering feeding solution comprising:
a reservoir having an outlet port;
a static mixing element disposed in fluid connection with the outlet port configured for mixing the solution; and
a pump means for delivering the solution.

54. The system of claim 52, wherein the mixing element comprises baffles for directing the flow of solution around the baffles to thereby mix the solution.

55. The system of claim 52, whereby the mixing element comprises plates having holes therethrough, and wherein the liquid is directed through the holes.

56. The method of claim 1, wherein the method further comprises utilizing a motor unit to agitate the bottle.

57. The method of claim 1, wherein the method further comprises passing the feeding solution through a static mixer.

58. The reservoir of claim 13, further comprising a static mixer.

59. The system of claim 15, further comprising a static mixer configured for mixing fluid exiting the reservoir.

60. The method of claim 16, wherein the method further comprises passing the feeding solution through a static mixer.

61. The method of claim 18, wherein the method further comprises passing the feeding solution through a static mixer after exiting the reservoir and before being delivered to an infant.

62. The method of claim 27, wherein the radially expandable container is formed from an elastomeric material.

63. The method of claim 27, wherein the radially expandable container applies pressure to the feeding solution.

64. The system of claim 30, further comprising a motor unit for agitating the reservoir.

65. The system of claim 30, further comprising a static mixer configured for mixing the feeding solution exiting the reservoir.

66. A system for delivering feeding solution comprising:
an elastically expandable reservoir comprising an inlet port and an exit port;
a static mixer disposed in fluid communication with the exit port; and
a pump for delivering the feeding solution.

67. A system for delivering feeding solution comprising:
a reservoir defining a generally planar surface being substantially smaller in thickness than in length or width, the reservoir comprising a plurality of walls disposed therein for dividing the reservoir into a tortuous pathway, an inlet port, and an outlet port; and
a pump for delivering feeding solution from the reservoir.

68. The system of claim 66, further comprising a static mixer disposed in fluid communication with the outlet port.
